(19) United States
(12) Patent Application Publication
(10) Pub. No.: US 2002/0119164 A1
(43) Pub. Date: Aug. 29, 2002

(54) Use of agaricus blazei murill to prevent or treat skin and other disorders

(76) Inventors: Shoji Uchiyama, Tokyo (JP); Kenji Haramaki, Palos Verdes Estates, CA (US)

Correspondence Address: CHRISTIE, PARKER & HALE, LLP, 350 WEST COLORADO BOULEVARD, SUITE 500, PASADENA, CA, 91105, US

(21) Appl. No.: 09/933,989

(22) Filed: Aug. 20, 2001

Related U.S. Application Data
(60) Provisional application No. 60/226,475, filed on Aug. 18, 2000.

(51) Int. Cl. A61K 35/84

(52) U.S. Cl. 424/195.15

(57) ABSTRACT

Agaricus blazei in whole, particulate, or extracted form, are useful as a barrier when applied to the skin against harmful effects of environmental toxins, pollution, chemicals, and radiation. Taken internally, whole, particulate, or extracted Agaricus blazei offer protection from various disorders such as autoimmune disorders, and alone or in conjunction with other therapies, can be beneficial to treat such disorders.

### CROSS-REFERENCE TO RELATED APPLICATION

This application is based on U.S. application Ser. No. 60/226,475, filed Aug. 18, 2000, the disclosure of which is incorporated by reference.

### FIELD OF INVENTION

The present invention is directed to a method of preventing or treating skin and other disorders in a subject. More specifically, the present invention is directed to a method of preventing and treating skin and other disorders in a patient by administering an effective amount of Agaricus blazei Murill.

### BACKGROUND OF THE INVENTION

Mushrooms have been used for medicinal purposes for centuries in many cultures. Several important drugs have been isolated from mushroom fruiting bodies and mycelium. Some examples of drugs obtained from mushrooms are lentinan from L. edodes, grifolin from Grifola frondosus, and krestin from Coriolus versicolor. These compounds are protein-bound polysaccharides or long chains of glucose, and are extracted from the cell walls. They have been used as anti-tumor immunomodulatory drugs. Administered orally or intravenously, they stimulate the body's immune system and enhance host-mediated resistance against some viral, bacterial, and fungal pathogens and various types of carcinomas. As immunostimulators, these drugs can enhance the activity of macrophages and T lymphocytes and raise interleukin-2 and antibody levels.

Mushrooms such as the above, and of the genus Agaricus, have been investigated for various uses, for instance, taken internally as antitumor agents, or to prevent skin diseases such as psoriasis and benign keratoses, to prevent aging (e.g., wrinkles and age spots), or to stimulate dermal fibroblasts.

Cancer is generally thought to result from one or more permanent genetic changes in a cell. It appears that the vast majority of human cancers arise as a result of the complex interplay between genetic and environmental factors. Environmental factors involved in the development of cancers can be chemical, physical, or biological carcinogenic agents. Environmental agents may act by inducing expression of oncogenes. Many chemical carcinogens are capable of inducing mutation, which can result in activation of the transforming potential of an oncogene. Agents that serve as promoters are often incapable of inducing cancers on their own, but significantly enhance the development of cancers by initiating agents. Viral carcinogenesis is widespread in nature. For example, some papilloma viruses are oncogenic in humans and are associated with skin cancer.

Chemical compounds capable of inducing cancer include polycyclic hydrocarbons, aromatic amines, and alkylating agents. Many chemical carcinogens are actually prodrugs activated to become carcinogenic by the body's metabolic machinery, such as the microsomal enzymes that evolved to detoxify toxic compounds. The active metabolite of many chemical carcinogens is a free-radical compound, and nearly all chemical carcinogens have been found to interact directly with DNA, forming adducts that can result in errors in base sequence during replication.

The three major physical carcinogens are ionizing radiation, ultraviolet radiation, and foreign bodies. Ionizing radiation can originate from many sources and can occur in many forms, but the two major categories are electromagnetic radiation, from x-rays and gamma rays, and particle radiation, originating in electrons, protons, neutrons, and alpha particles. Radiation may induce cancers at any site. More than 80% of radiation exposure is from natural sources such as cosmic rays, terrestrial gamma rays, and radon. Radon may emanate from the ground and from building materials, disperse in the air, and decay into short-lived aerosolized alpha particles. Medical uses of radiation account for about 18% of the total radiation exposure.

Ultraviolet radiation, mainly from the sun, is carcinogenic to skin. The incidence of skin cancers other than melanoma is much higher in southern latitudes and on sites exposed to the sun, and the incidence of melanoma may also be augmented. The UVB portion (280-320 nanometers) of the ultraviolet spectrum is most damaging to tissues. Ultraviolet radiation induces the formation of pyrimidine dimers (usually between thymines), which brings about alterations in the normal sequence of bases in DNA. Ultraviolet radiation also exerts systemic effects by altering the immune function.

Lifestyle diseases such as cancer, diabetes, heart disease and liver disease have become increasingly common due to factors such as changes in diet, a deterioration in the environment, and pollution. Additionally, hay fever, atopy, and allergies continue to increase, especially among young people. Many cancers, neoplasms, and similar disorders are difficult to treat, or involve the use of drugs with undesirable side effects (for example, steroids).

Accordingly, there exists a need for a simple, effective preventative and treatment for these ailments.

### SUMMARY OF THE INVENTION

The extracts and methods of the invention provide a safe, natural, and easy way to help prevent and treat skin disorders induced by carcinogenic environmental factors, and autoimmune disorders. The raw material for the invention is a basidiomycetous fungus, Agaricus blazei. The invention provides methods of using Agaricus blazei to treat or prevent a skin cancer in a patient, which involves using whole, particulate or extracts of the Agaricus mushroom and contacting the mushroom with the skin of the patient. Preferably, the Agaricus is provided in an easy to apply form, such as in a cream or lotion, and can be mixed with other active or inactive ingredients (e.g., sunscreen, vitamins, or herbal extracts). The Agaricus protects the skin against damage caused by pollution and environmental chemical exposure (such as emission and exhaust from automobiles, pesticides, etc.) or from radiation (for example, UV radiation from sun exposure).

In another embodiment, the Agaricus is taken internally to treat or prevent disorders such as autoimmune disorders or disorders induced by carcinogenic environmental factors. The Agaricus may be taken alone or mixed with other active or inactive ingredients, and may be provided in any form suitable for oral administration.

### DESCRIPTION OF THE DRAWINGS

FIG. 1 is a schematic of the protocols used to produce the Agaricus extract according to one embodiment of the present invention.

FIG. 2 is a schematic of the separation procedure and products obtained from the Agaricus according to the present invention.

FIG. 3 is a schematic of the protocol used to evaluate oral application of an Agaricus aqueous solution on tumor promotion and growth.

FIG. 4 is a schematic of the protocol used to evaluate oral application of an Agaricus aqueous solution on tumor promotion and growth.

FIG. 5 shows the results of the experiment of FIGS. 3 and 4. (A) percentage of mice bearing papillomas; (B) average number of papillomas per mouse. Filled circles are controls, open circles were given 0.0025% Agaricus solution in their water.

FIG. 6 shows the results of the experiment of FIGS. 3 and 4. (A) percentage of mice bearing papillomas; (B) average number of papillomas per mouse. Filled circles are controls, open circles were given 0.0025% Agaricus solution in their water.

FIG. 7 is a schematic of the protocol used to evaluate an oral application of an Agaricus aqueous solution on UVB induced tumor promotion and growth.

FIG. 8 is a schematic of the protocol used to evaluate an oral application of an Agaricus aqueous solution on UVB induced tumor promotion and growth.

FIG. 9 is a schematic of the protocol used to evaluate an administration of a topical application of Agaricus aqueous solution on UVB induced tumor promotion and growth.

FIG. 10 is a schematic of the protocol used to evaluate an administration of a topical application of Agaricus aqueous solution on UVB induced tumor promotion and growth.

FIG. 11 shows the results of the experiment of FIG. 10. (A) percentage of mice bearing papillomas; (B) average number of papillomas per mouse. Filled circles are controls, open circles were given 50 μg of Agaricus topically.

FIG. 12 shows the results of the experiment of FIG. 10. (A) percentage of mice bearing papillomas; (B) average number of papillomas per mouse. Filled circles are controls, open circles were given 50 μg of Agaricus topically.

### DETAILED DESCRIPTION OF THE INVENTION

The present invention uses the whole or particulate mushroom Agaricus blazei and extracts thereof to prevent and treat forms of damage to skin caused by environmental toxins and carcinogens. Such damage includes neoplasms, skin cancers, damage from ultraviolet radiation, and chemical damage. It has been found that, when applied to the skin, this mushroom and extracts thereof offer protection from environmental pollutants, toxins, and carcinogens (such as found in automobile emissions) that attack the skin, and are palliative, alone or in combination with other treatments, for conditions caused by such pollutants, toxins, and carcinogens. When these components are extracted with water and contacted with the skin, alone or with other active ingredients, many such skin disorders can be prevented or treated in humans and animals.

It has also been discovered thatAgaricus blazei contains component(s) which, when taken internally, are effective to treat or prevent disorders such as diabetes and autoimmune disorders such as rheumatoid arthritis, certain thyroid conditions, and lupus erythematosus. Unlike previous studies with mushrooms showing enhanced immune function, Agaricus blazei and its extracts can downregulate immune function in cases of autoimmune disorders.

Mushrooms of the species Agaricus blazei have been cultivated, but are indigenous to the Piedate mountain region outside Sao Paulo Brazil. Cultivation is difficult, because the mushroom requires conditions similar to its native Piedate mountains, where there is a temperature variance between 20° C. and 35° C. daily, and an average humidity of about 80%. Commercial sources exist in Japan, Brazil, China, and the United States. A preferred source of Agaricus blazei is Sylvan, Inc., Saxonburg, Pa. This commercial source is particularly preferred because of the well controlled environment in which the mushrooms are cultivated, leading to a reliable product batch to batch. Of particular concern are the following factors:

1) Purity of the fungus—few or no contaminating bacteria, other fungi, or other ingredients.

2) Growth conditions—uniform temperature and moisture conditions provide reliable growth, life cycle, and morphology characteristics.

3) Nutrient and chemical composition—uniform soil and compost conditions provide fungus with reliable quantities of various active compounds that do not vary between batches.

4) Controlled quantities of heavy metals—consistent growth media lead to mushrooms with lower content of toxic heavy metals such as mercury, cadmium, and arsenic, and controlled content of copper, manganese, etc.

A. blazei can be used fresh or dried, and are extracted with a water or aqueous solution for use in the invention. By “water extraction” is meant that certain components are solubilized by any aqueous solvent. The extraction can be done by immersing mushrooms or parts thereof (e.g., spores, cap (pileus), stipe, annulus, gill/lamellae, basidia, filaments), in any or all stages of the life cycle, whether whole or particulate (e.g., chopped or ground to powder), in an aqueous solution (including pure water). The temperature of the aqueous solution can be varied to alter the amount or type of components extracted, but is preferably between about 26° C. and boiling (about 100° C.), more preferably over about 90° C. The aqueous solution in which the mushrooms have been extracted is preferably used, but the moistened mushrooms, if not fully extracted, can also be used topically or internally.

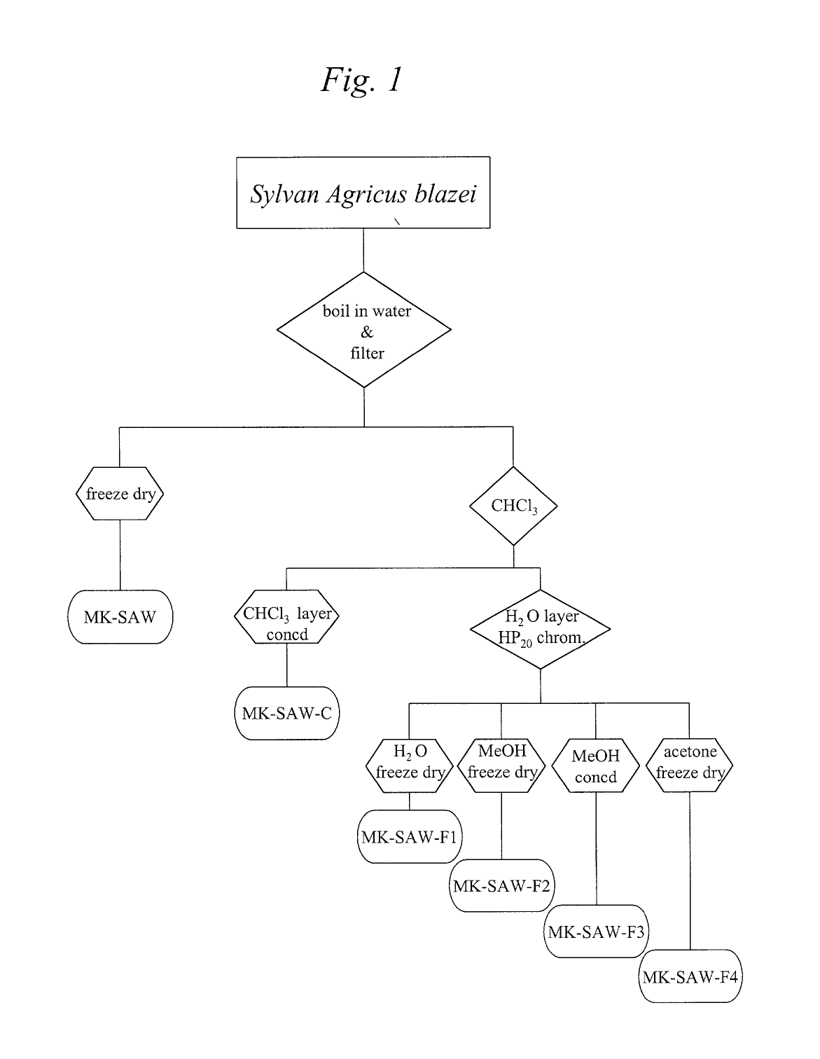

Any aqueous extraction protocol can be used, but a preferred extraction procedure is given in Japanese Publication JP 09315994A, which includes hot water extraction and further extraction of the residue with 5% aqueous solution of ammonium oxalate, then decomposing the extract with hydrochloric acid and subjecting the material to gel permeation and purification with affinity chromatography. A more preferred extraction method is a plain hot water extraction, comprising exposing whole or particulate Agaricus blazei to hot water, preferably over 90° C., for at least 30 minutes, with at least occasional agitation or stirring. Preferably, the ratio of mushroom to water is between about 1:2 and 1:100 (wt/wt), more preferably between about 1:2.5 and 1:25 (wt/wt). Ratios depend on the planned use of the extract, for example, it is preferred to use about 1 part by weight dried mushroom to 50 parts by weight water for internal use, and 1 parts by weight dried mushroom to 2.67 parts by weight of water for use in skin treatment compositions. Optionally the products may be freeze dried or concentrated using water or methanol or acetone solutions. A flow-chart of these various extraction methods is shown in FIG. 1.

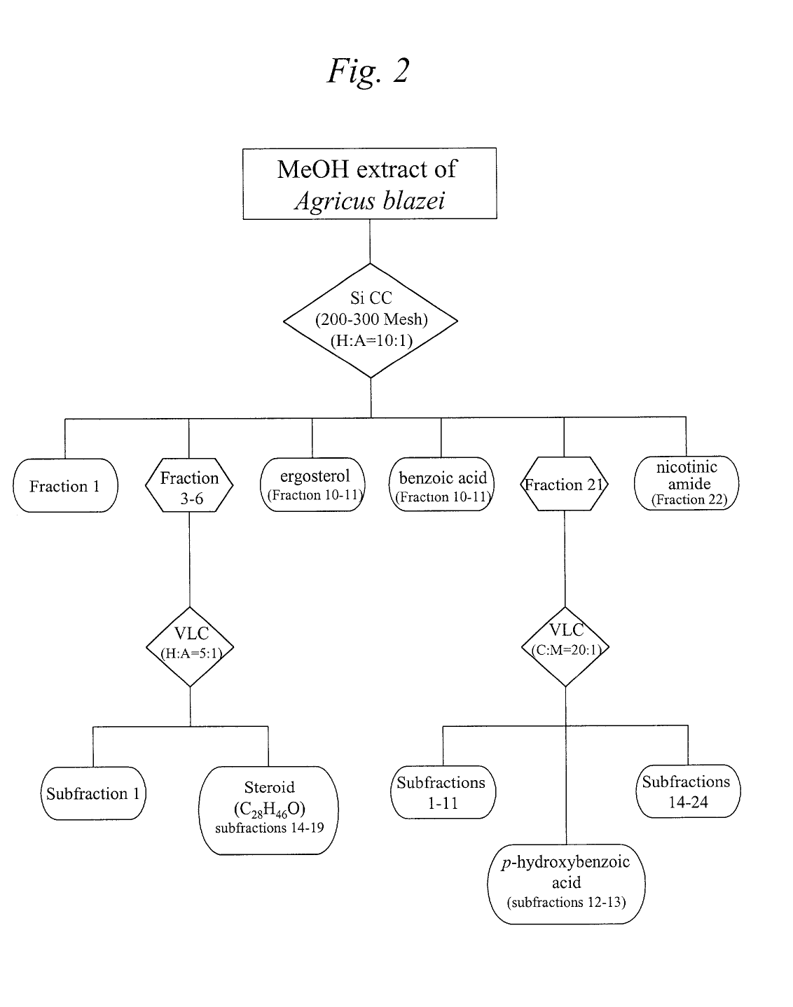

Identified compounds that have been extracted from Agaricus blazei include polysaccharide-glucan, (exhibits anti-tumor and blood glucose reduction effects through actions on, e.g., NK cells, T cells, macrophages and other immune cells, possibly through regulation of interferon release); steroids (exhibit anticancer effects and effects on osteoporosis through inhibition of tumor cell proliferation and vitamin D2 regulation); dietary fiber and linoleic acid (aid in prevention of high blood pressure, arterial sclerosis, and improvement of hepatic disfunction); ergosterol; nicotinic acid amide; benzoic acid; beta glucans; and others. FIG. 2, illustrates the separation procedure and the fractions obtained thereby. As shown, the above compounds can be obtained controllably and reproducibly in different fractions and subfractions of a methanol extract of the Agaricus blazei mushroom.

Although only extracts of Agaricus blazei are discussed above, any form of Agaricus blazei incorporating the active ingredients described above may be utilized in the present invention. For example, whole or particulate mushrooms in any form may be utilized to provide the necessary active ingredients. In one embodiment of the current invention whole Agaricus blazei is ground into a powder and mixed into a topical or oral formulation to provide the therapeutic properties of the current invention.

The above manufacturing techniques may utilize any portion and any life stage of the Agaricus blazei. For example, the mushroom cap and/or stem may be used in any of the formulations of the invention. In addition, any or all of the four life stage of the mushroom: spore, mycelium, primordial and fruit body may be used to make the formulations of the present invention. In one preferred embodiment, all Agaricus blazei mushrooms in all four life stages are used together in a single formulation.

Skin Treatments

Examples of skin conditions that may be prevented or treated by using compositions of the invention include the following.

Skin neoplasms may be benign or malignant, congenital or acquired, and they may arise from any component of the skin. The common mole (melanocytic nevus) is a neoplasm of benign melanocytes; usually it is acquired, but it may be present at birth, when it is often known as a birthmark. Certain neoplasms, such as giant congenital melanocytic nevi, can become malignant and can be treated with the compositions of the invention to prevent malignancy.

Malignant neoplasms may arise from cellular elements of the epidermis or dermis, or by infiltration of the skin by malignant cells arising from other tissues. The most common are basal cell and squamous cell cancers, which arise from basal and squamous keratinocytes of the epidermis, respectively. These constitute the most common form of cancer in the United States, with over 600,000 new cases per year. They are caused by the cumulative effects of ultraviolet radiation on the skin. They are locally invasive in almost all cases, rarely metastasize or cause death, and are readily recognized. They usually are characterized by a non-healing sore, persistent red scaling or crusting patch, or a slowly growing pearly nodule on skin that has been exposed to the sun; they occur mostly on the head, neck, hands, and arms. Additionally, some papilloma viruses are associated with cervical cancer and skin cancer. The compositions of the invention, topically or orally administered, are suitable for use to prevent or treat such lesions and they may be used with any other therapies. They are sufficiently easy to use that patient compliance is high.

Malignant melanoma arises from the pigment-forming melanocyte, and thus it is usually pigmented. Other signs and symptoms, such as bleeding, pain, and itching, are less frequent, often occurring as late manifestations. Malignant melanoma is one of the most rapidly increasing cancers, probably related to increased ultraviolet radiation exposure. Extracts of Agaricus blazei according to the invention aid in preventing the occurrence of melanoma from such environmental radiation exposure.

Two unusual multicentric primary skin malignancies are mycosis fungoides and Kaposi's sarcoma. Mycosis fungoides is a lymphoma of the skin, usually present in several sites when first diagnosed, that may remain confined to the skin for 10 or more years before eventually spreading to internal organs and causing death. Kaposi's sarcoma is usually not fatal, although it may eventually spread to internal organs and may cause significant morbidity. There are numerous other primary skin cancers, which occur less frequently.

Examples of viral diseases that affect the skin include viral warts (verruca vulgaris) and molluscum contagiosum. Both are characterized by single or multiple, somewhat contagious, skin tumors that usually are small but can in rare instances exceed 0.4 in. (1 cm) in diameter.

Prevention and treatment of such conditions by application of compositions and methods of the invention can be as follows. The extract for use in skin treatments is preferably extracted at a ratio of about one part mushroom by weight to 2-5 parts aqueous solution using extraction methods described above. This extract can be used directly on the skin (at 100% strength), but is preferably put into a formulation for use on the skin in a concentration of at least 0.01% vol/vol, preferably at least 0.05%, and most preferably between about 0.05% and 50%. Although specific volume concentrations are provided above, any composition suitable for preventing or treating disorders in a user may be utilized in the present invention. The balance of ingredients in such compositions can be other active or inactive ingredients, depending on the properties of the composition desired. For example, other ingredients can be such things as sunscreens, glycolic acid, colostrum, vitamin C or other vitamins, herb extracts such as chamomile or lavender (available from sources such as Pacific Research, Inc. TX), and suitable excipients or emollients.

The extracts of the invention are preferably compounded with other active and/or inactive ingredients to make application easy and pleasant, or to provide other therapeutic or aesthetic advantages. Such products are preferably in the form of body or face creams or lotions, but can also include a variety of other products, such as products for use in the bath These products would include products where the whole or particulate mushroom or its extracts are added to a disposable porous material bag to be placed into bathwater, or added to compressed cubes or other physical forms of powder and gel, including effervescent blocks; bath gel; scented bath and body oils; bubble bath; bath salts or crystals, combined, e.g., with sea salts or other minerals; body wash; body scrub (exfoliators); soft-capsule “pearls” or balls; soap of any type, including liquid, bars, granules or flakes; liquid gels for children that tint the water, or tub gel “paints” (e.g., soap-based); and aromatherapy additives.

Products for topical application include adding the mushroom or its extracts to spritzers and sprays, e.g., perfume, eau de parfum; body splash (light fragrance); scented and unscented body lotions and emollients, including moisturizers, ointments, greases, skin softeners; body oils, powders, gels, purees or emulsifications of various natural items like fruit and herbs; shampoos, conditioners and creme rinses; body muds and masks; foam (e.g., shaving creams); cooling gels; make-up removal creams; tanning gels, creams, and lotions, including sunscreen-containing products or self-tanning products; lip balm; cosmetics; astringents, moisturizers, exfoliators, makeup removers and nail care products, such as polish, cuticle oil, polish remover; generally, any products containing “slip” (a binder that allows pigment to slide across the skin); and any other product that softens the skin or soothes irritation in the skin.

To gain the maximum protective and therapeutic benefits of the extracts of Agaricus blazei, whether alone or mixed with other ingredients, the extract should be applied at least once a day, particularly before exposure to pollutants such as automobile emissions, but can be applied as often as desired (for example, after swimming or bathing), or as infrequently as desired.

Internal Treatments

Failure of immune tolerance to self constituents results in an autoimmune response which is often associated with autoimmune disease. Autoimmune disease occurs when the autoimmune response to self constituents has damaging effects of a structural or functional character. Failure of immune regulation is responsible for autoimmune disease. Many human diseases can be attributed to autoimmune reactions. Circulating autoantibodies are responsible for diseases in which there is intravascular destruction, for example, the red blood cells in hemolytic anemia. T lymphocytes may be responsible for some types of thyroid goiter, such as Hashimoto's disease; a stomach mucosal degeneration that results in nonabsorption of vitamin B 12 and thus the blood disease pernicious anemia; the insulin-dependent or juvenile type of diabetes mellitus; and one type of chronic hepatitis. Immune complexes cause glomerulonephritis and most of the features of systemic lupus erythematosus, in which autoantibodies are formed to various constituents of cell nuclei. In Sjongren's disease, in which salivary and lacrimal glands are destroyed, damage by T lymphocytes within the glands may be accompanied by damage by immune complexes throughout the body. Some autoimmune diseases are caused by antibodies to cell receptors, which either block neuromuscular transmission, as in myasthenia gravis, or stimulate thyroid cells to overactivity, as in Graves' disease. Some important human diseases may be autoimmune disorders, including rheumatoid arthritis, multiple sclerosis, and ulcerative colitis.

To prevent and treat conditions such as diabetes, lupus, rheumatism, certain types of cancer, etc., with the extracts and methods of the invention, extraction of between about 1:25 and 1:100 mushroom to water (wt/wt) is preferred, more preferably in weight of dried mushrooms or particulates. Alternatively, whole or particulate mushrooms can be eaten (for extraction by digestion, or soaked in water to make a tea for consumption. This treatment can be used alone or in combination with any other therapy. It has the advantage of being a natural, safe, and nutritious therapy when taken internally, and does not interact with pharmaceutical compounds that are taken concurrently. In alternative forms the Agricus blazei may be put in the form of actual food products or in dietary supplements.

The extract can be formulated as a liquid, capsule, or pill for oral use, using any excipients or methods known to those of skill in the pharmaceutical arts, for example using methods disclosed in Remington: The Science and Practice of Pharmacy, 19th ed. (1995) Mack Publishing Company, Easton, Pa.; herein incorporated by reference. Extract to be used in certain forms (e.g., tablets), will generally be dried before compounding with excipients to make that formulation. Compressed tablets can be formed by compression and may or may not be coated (e.g., with sugar, film, time-release, or enteric coatings). They are made from powdered, crystalline, or granular Agaricus material or dried extract, alone or in combination with binders, disintegrants, controlled-release polymers, lubricants, diluents, flavorings and colorants. Gelatin capsules can be filled with powdered, crystalline, or granular Agaricus material or dried extract. Solutions, emulsions, suspensions, and extracts for internal or external use may be prepared by dissolving the Agaricus blazei extract in an aqueous or nonaqueous solvent, using, for example, ingredients such as mineral oil, fish oils, or fruit, spice or vegetable oil; ethanol; water; glycerin; sorbitol; propylene glycol; flavoring agents; preservatives; and syrups. Creams and lotions for external use are intended to mean liquid or semiliquid preparations that contain one or more active ingredients in suitable excipients, and are generally suspensions of solids in an aqueous medium. A wide variety of ingredients may be added to the preparation to produce better dispersions or to accentuate cooling, soothing, drying, or protective properties. Bentonite is an example of a suspending agent used in the preparation of lotions. Methylcellulose or sodium carboxymethylcellulose, for example, will hold the active ingredient in contact with the skin and is also easy to rinse off with water. A formulation containing glycerin will keep the skin moist. Alcohols will add a drying and cooling effect.

Although the above embodiments primarily discuss formulations for humans, the formulations may also be directed to preventing and treating disorders in domestic or livestock animals. For example, the Agaricus blazei could be mixed with a meal base to form animal food, such as domestic animal pet food or livestock feed. Alternatively, the Agaricus blazei could be formulated into a pet or livestock medication or dietary supplement.

As with the topical compositions, additional appropriate active and inactive ingredients may be used, such as herbal extracts and/or vitamins such as vitamin C. Appropriate dosages for maximum therapeutic benefit are preferably equivalent to an amount extracted from between about 2 g to about 10 g of dried Agaricus blazei per day. Larger or smaller doses can be taken as well without adverse affect. The extract can be taken daily in one or more doses, but a preferred regimen of dosing is a 10 to 30 day course of therapy, preferably with intervals of between 1-10 days on the extract followed by 1-10 days off of the extract.

### EXAMPLE 1

### Cytotoxicity of Agaricus blazei

The cytotoxicity of eight different preparations of Agaricus balzei made using the extraction techniques described in FIG. 1 were tested utilizing a sulforhodamine B microtitre plate assay, according to the procedure described in: J.N.C.O., 82: 1107-1112 (1990), incorporated herein by reference. In this procedure, samples of the various extracts were assayed over three days for cytotoxicity to a variety of human tumor cell lines, including: Glioelastoma (HTCL: U-87-MG); bone (HTCL: HOS); breast (HTCL: MCF-7); and melanoma (HTCL: SK-MEL-2). The tumor cells utilized were cultured in an RPMI-1640 growth medium, supplemented with 25 mM HEPES, 2% (w/v) sodium bicarbonate, 10% (v/v) fetal bovine serum and 100 μg/mL kanamycin. The cultures were maintained in a humidified atmosphere of 5% carbon dioxide at 27° C. The cells were then treated with duplicate treatments of 20 and 10 μg/mL of the screen compound extract. The interpolated ED₅₀ values from the dose-response curves generated from the assays are reported in Table 1, below.

TABLE 1
Cytotoxicity Results

| | Glioelastoma | Bone | Melanoma | Breast |
|---|---|---|---|---|
| | ED₅₀ | ED₅₀ | ED₅₀ | ED₅₀ |
| Extract ID | (μg/mL) | (μg/mL) | (μg/mL) | (μg/mL) |
| MK-SAW | >20(10) | >20(8) | >30(23) | >20(18) |
| MK-AJW | >20(22) | >20(14) | >20(23) | >20(28) |
| MK-SAW-C | Not Active | Not Active | >20(16) | >20(8) |
| MK-SAW-F1 | >20(6) | Not Active | >20(10) | >20(13) |
| MK-SAW-F2 | Not Active | Not Active | Not Active | Not Active |
| MK-SAW-F3 | Not Active | >20(5) | Not Active | >20(13) |
| MK-SAW-F4 | >20(12) | >20(18) | >20(14) | >20(27 |

In addition, the extracts above were tested in vitro to determine their biological activities relative to each other and to the control. Comparing the viability of the tumor cells in these samples treated with the test extract with the tumor cell control sample allows for a calculation of the relative ratio of the extract activation with respect to the positive control, as shown in Table 2, below.

TABLE 2
Relative Ratio of Activation With Respect to Control

| | 100 μg/mL | | |
|---|---|---|---|
| | Extract | | |
| | % To Control | 10 μg/mL Extract | 1 μg/mL Extract |
| Extract ID | (% Viability) | % To Control | % To Control |
| MK-SAW | 18.6(80) | 59.3 | 97.7 |
| MK-SAW-C | 9.3(60) | 18.9 | 90.6 |
| MK-SAW-F1 | 31.6(70) | 69.2 | 100 |
| MK-SAW-F1˜2 | 22.2(70) | 64.9 | 100 |
| MK-SAW-F2 | 18.5(80) | 51.3 | 93.7 |
| MK-SAW-F3 | 13.3(80) | 50.7 | 91.3 |
| MK-SAW-F4 | 12.2(60) | 50.3 | 91.1 |

In this test a solution of EBV genome-carrying, non-producer Raji cells were treated with 4 mMol of n-butyric acid and 20 ng of a tumor producing agent (TPA) to activate tumors therein. The cells were then dosed with a solution of the test compound in DMSO. The cells were then cultured as described above in a carbon dioxide incubator for 48 hours at 37° C. The cells were then smeared and the smears stained by an NPC serum for the EBV-EA producing cells by indirect immunofluorescence.

According to the above results, then, the extract of CHCl₃ has the greatest active inhibitory effect among the extracts tested. The results indicate that the order of activity of the most to least active of the extracts is as follows: MK-SAW-C>F4>F3>F2>SAW>F1>F1-2. However, all of the extracts showed greater inhibitory effect than did the control sample indicating that the Agaricus blazei extract can be used to inhibit a variety of tumor cells.

### EXAMPLE 2

### Anti-Tumor Effects of An Oral Application of Agaricus blazei

The table below shows that Agaricus blazei Murill produced good results for both complete recovery and for anti cancer effect. The experiment used mice of between five and six weeks old. Vaccinating sarcoma 180 (a type of cancer cell) into the femur of these mice normally causes cancer to spread to the entire body over four to five weeks, resulting in the death of almost all these animals. The fungus extract was first administered 24 hours after vaccination, when the cancer cells were firmly embedded in the animals' tissues, and the process continued for 10 consecutive days. The results were then recorded four to five weeks later. The experiment was repeated on groups of between five and ten mice, which were each given a different fungus extract. The mean values taken from these experiments were expressed as percentages.

The anti cancer effect rate represents the percentage of mice which fully recovered from the cancer induced by an initial vaccination of sarcoma 180 and in whom a second vaccination of sarcoma 180 failed because the cancer cells could not be successfully embedded.

From these results, it was deduced that the fungus extract (component primarily comprising a high-molecular polysaccharide) activates the immunity of normal biological tissue, so that even when a virus or other external factors enter the tissue, macrophage and interferon production within the tissue is vitalized to prevent the multiplication, metastasis and reoccurrence of cancer cells. The results of this test including the daily dosage for each of the administered materials, the rate of complete recovery and the “anti cancer” effect for each of the materials is provided in summary in Table 3, below.

TABLE 3
Comparison of Anticancer Effectiveness of Various Fungi

| | | Rate of complete | Anti cancer |
|---|---|---|---|
| Name of fungus | Daily dosage | recovery | effect |
| Agaricus blazei Murill | 10 mg | 90.0% | 99.4% |
| Grifola umbellata | 10 mg | 90.0% | 98.5% |
| Phellinus yucatensis | 30 mg | 87.5% | 96.5% |
| Phellinus igniarius | 30 mg | 66.7% | 87.4% |
| Lenzites betulina | 30 mg | 57.1% | 70.2% |
| Tricholoma matsutake | 30 mg | 55.5% | 91.3% |
| Lentinus edodes | 30 mg | 54.5% | 80.7% |
| Coriolus versicolor | 30 mg | 50.0% | 77.5% |
| Pleurotus ostreatus | 30 mg | 45.5% | 75.3% |
| Elfringia applanata | 30 mg | 45.5% | 64.9% |
| Fomitopsis pincicola | 30 mg | 33.3% | 61.2% |
| Fomitopsis cytisna | 30 mg | 30.3% | 44.2% |
| Pholiota nameko | 30 mg | 30.0% | 86.5% |
| Flammulina velutipes | 30 mg | 30.0% | 81.1% |
| Ganoderma lucidum | 30 mg | 20.0% | 77.8% |

### EXAMPLE 3

### TPA Promoted Tumor Inhibition by Oral Water-Extracted Agaricus

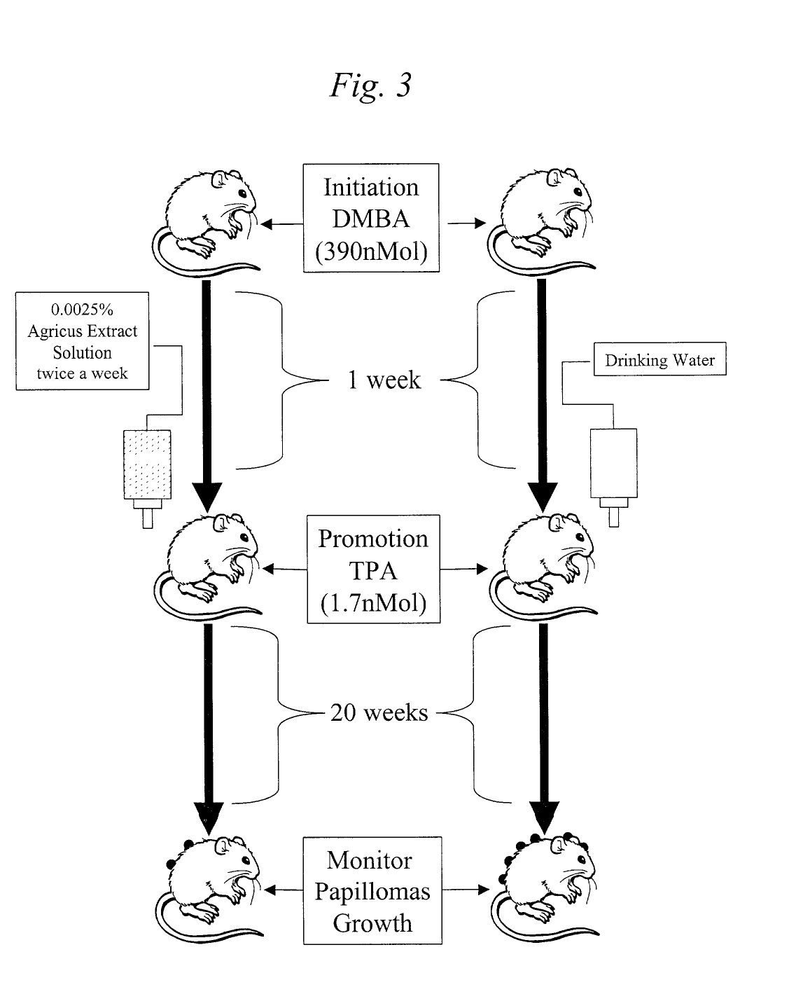

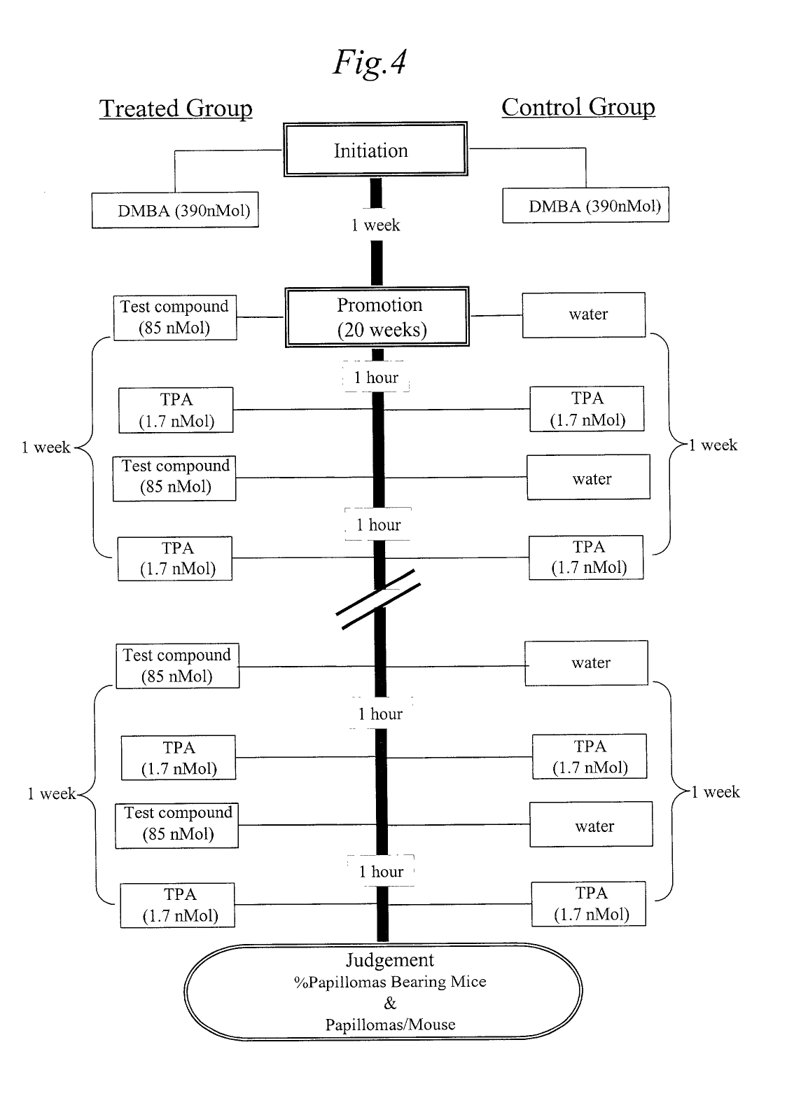

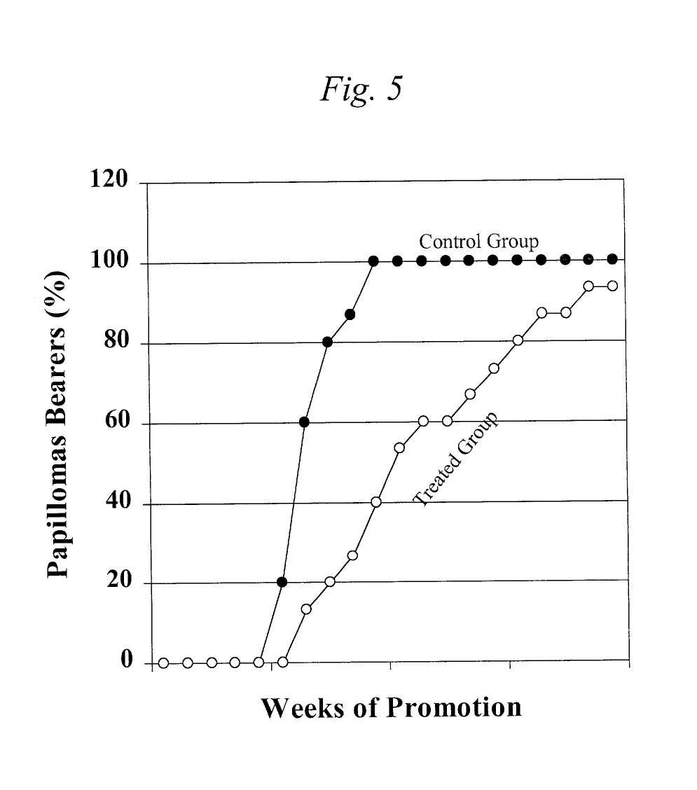

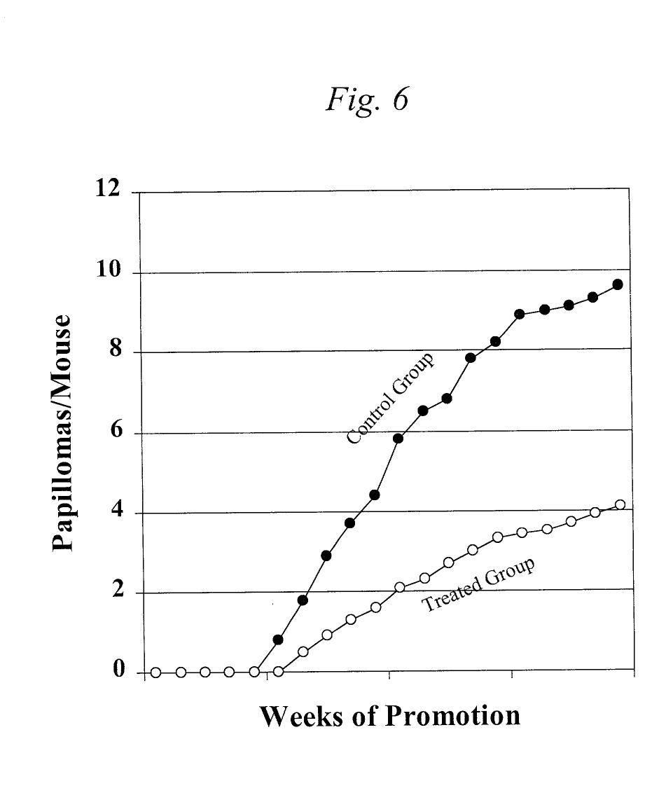

Tumors were initiated in mice with DMBA (390 nmol) and promoted with 1.7 nmol of TPA given twice weekly starting 1 week after initiation. Mice to be treated were given 0.0025% Agaricus aqueous extract in their drinking water and compared to control mice initiated and promoted in the same way but given normal drinking water (FIGS. 3 and 4). Table 4, below and FIGS. 5 and 6 show the inhibitory effects of orally administered Agaricus aqueous extract on tumor proliferation. At 20 weeks of promotion, controls significantly differed from the treated group in papillomas per mouse (p>0.05) and in the number of mice showing the presence of papillomas.

TABLE 4
Test Results for Oral Treatment of TPA Promoted Mice

| Positive control | | | 0.0025% Agaricus extract | |
|---|---|---|---|---|
| DMBA (390 nmol) + TPA (1.7 nmol) | | | (oral) | |
| | | Papillomas/ | | Papillomas/ |
| Week | Papillomas (%) | Mouse | Papillomas (%) | Mouse |
| 1 | 0 | 0 | 0 | 0 |
| 2 | 0 | 0 | 0 | 0 |
| 3 | 0 | 0 | 0 | 0 |
| 4 | 0 | 0 | 0 | 0 |
| 5 | 0 | 0 | 0 | 0 |
| 6 | 20 | 0.8 | 0 | 0 |
| 7 | 60 | 0.8 | 13.3 | 0.5 |
| 8 | 80 | 2.9 | 20 | 0.9 |
| 9 | 86.6 | 4.7 | 28.6 | 1.3 |
| 10 | 100 | 4.4 | 40 | 1.6 |
| 11 | 100 | 5.8 | 53.0 | 2.4 |
| 12 | 100 | 6.5 | 60.0 | 2.8 |
| 13 | 100 | 6.8 | 60.0 | 3.7 |
| 14 | 100 | 7.8 | 66.6 | 3.0 |
| 15 | 100 | 8.2 | 76.3 | 3.3 |
| 16 | 100 | 8.9 | 80 | 3.1 |
| 17 | 100 | 9.0 | 86.6 | 3.5 |
| 18 | 100 | 9.1 | 86.6 | 3.7 |
| 19 | 100 | 9.3 | 93.3 | 3.9 |
| 20 | 100 | 9.6 | 93.3 | 4.1 |

### EXAMPLE 4

### UVB Promoted Tumor Inhibition by Oral Water-Extracted Agaricus

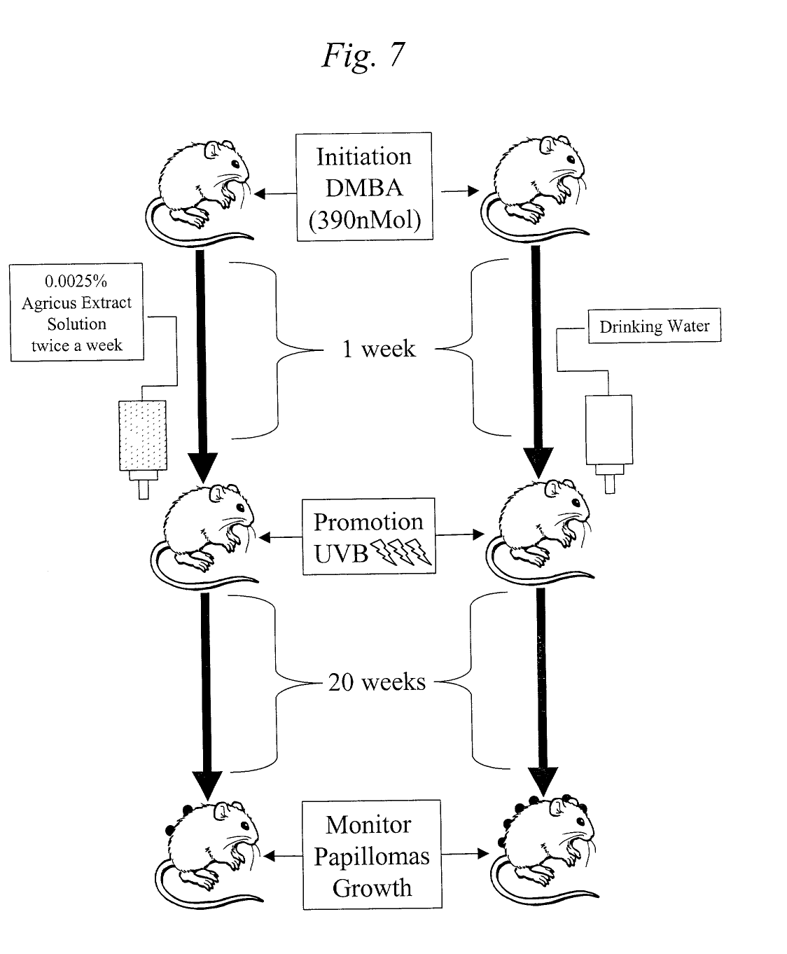

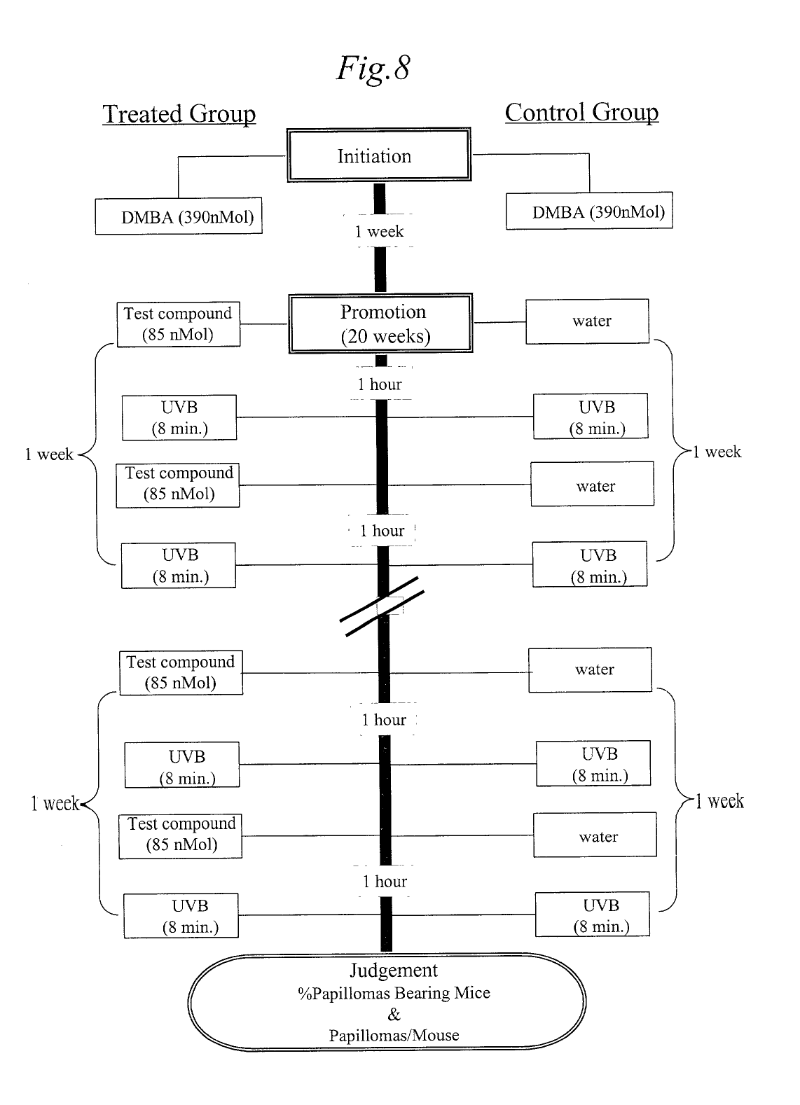

In a second experiment tumors were initiated in mice with DMBA (390 nmol) and promoted with eight minute exposures of 3430 J/m² UVB light given twice weekly starting 1 week after initiation. Mice to be treated were given 0.0025% Agaricus aqueous extract in their drinking water and compared to control mice initiated and promoted in the same way but given normal drinking water (FIGS. 7 and 8). Table 5, below shows the inhibitory effects of orally administered Agaricus aqueous extract on tumor proliferation.

TABLE 5
Test Results for Oral Treatment of UVB Promoted Mice

| Positive control | | | 0.0025% Agaricus extract | |
|---|---|---|---|---|
| DMBA (390 nmol) + UVB (2x/week) | | | (oral) | |
| | | Papillomas/ | | Papillomas/ |
| Week | Papillomas (%) | Mouse | Papillomas (%) | Mouse |
| 1 | 0 | 0 | 0 | 0 |
| 2 | 0 | 0 | 0 | 0 |
| 3 | 0 | 0 | 0 | 0 |
| 4 | 0 | 0 | 0 | 0 |
| 5 | 0 | 0 | 0 | 0 |
| 6 | 0 | 0 | 0 | 0 |
| 7 | 20 | 0.3 | 0 | 0 |
| 8 | 60 | 0.8 | 6.6 | 0.2 |
| 9 | 80 | 1.2 | 20 | 0.4 |
| 10 | 86.6 | 1.6 | 26.6 | 0.5 |
| 11 | 100 | 1.9 | 33.3 | 0.6 |
| 12 | 100 | 2.1 | 46.6 | 0.7 |
| 13 | 100 | 2.3 | 46.6 | 0.9 |
| 14 | 100 | 2.6 | 53.3 | 1.1 |
| 15 | 100 | 2.9 | 66.6 | 1.3 |
| 16 | 100 | 3.2 | 73.3 | 1.5 |
| 17 | 100 | 3.5 | 80 | 1.6 |
| 18 | 100 | 3.8 | 86.6 | 1.8 |
| 19 | 100 | 4.0 | 93.3 | 1.9 |
| 20 | 100 | 4.3 | 93.3 | 2.0 |

At 20 weeks of promotion, controls significantly differed from the treated group in papillomas per mouse (p>0.01) and in the number of mice showing the presence of papillomas.

### EXAMPLE 5

### UVB Promoted Tumor Inhibition by Topical Water-Extracted Agaricus

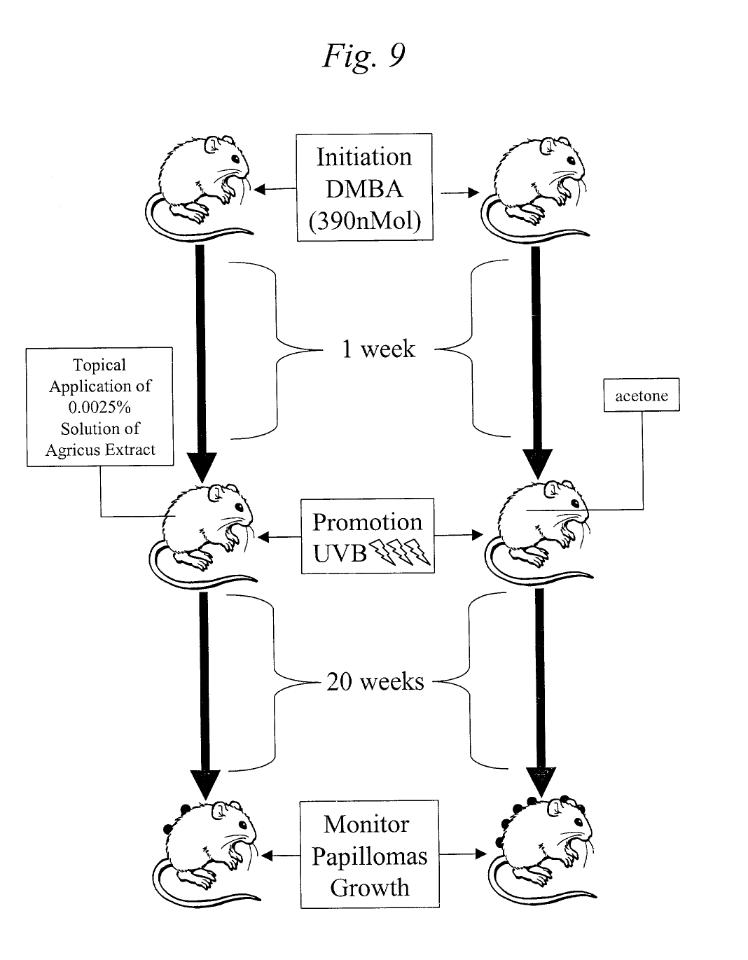

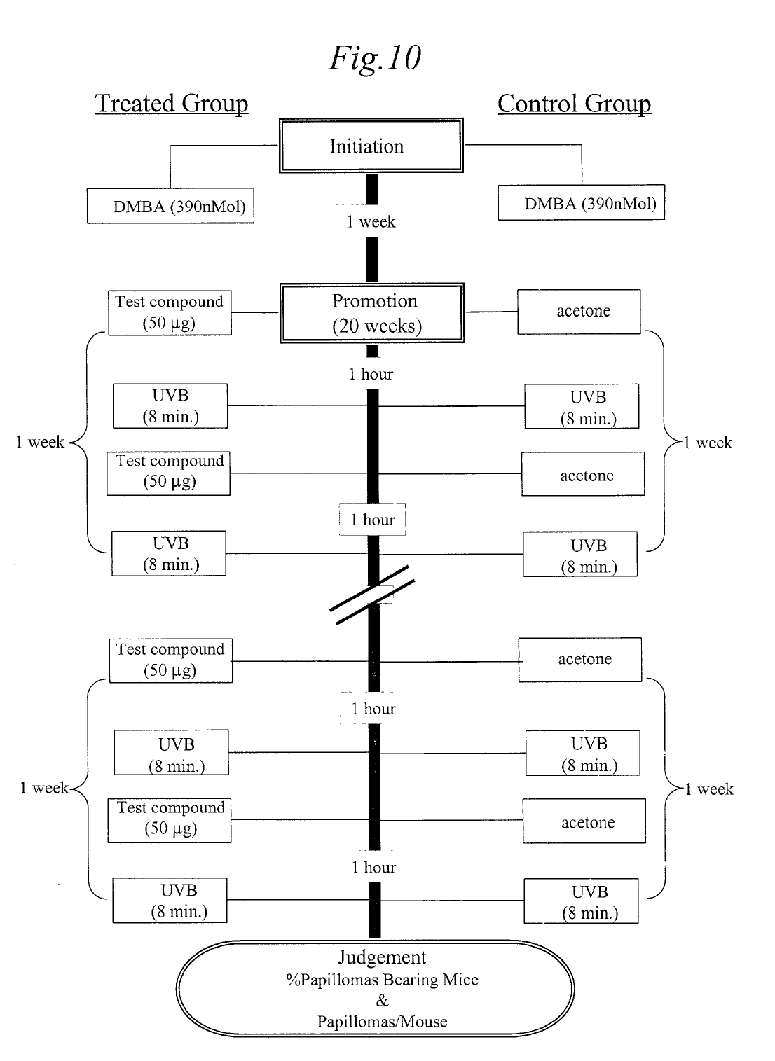

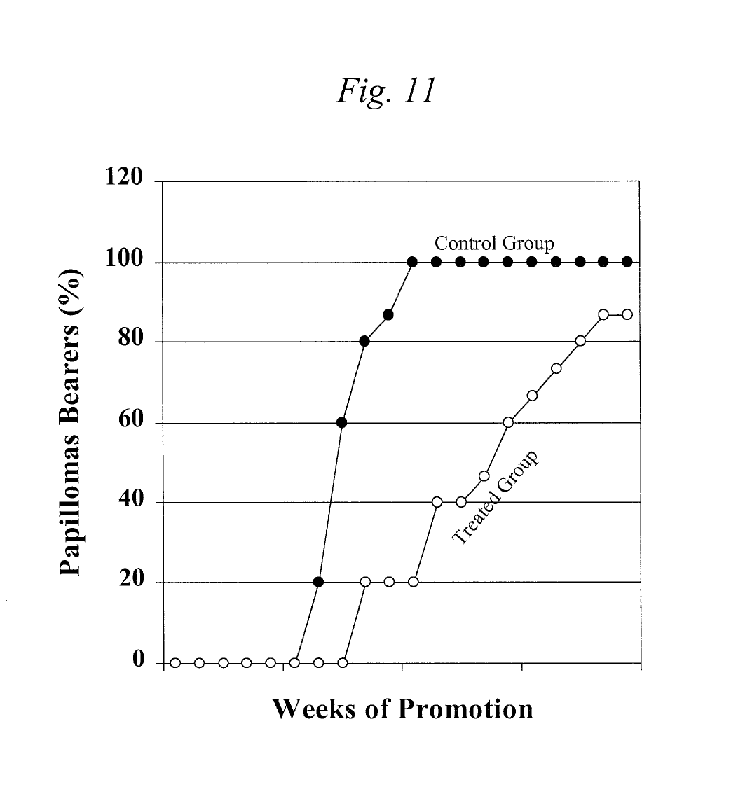

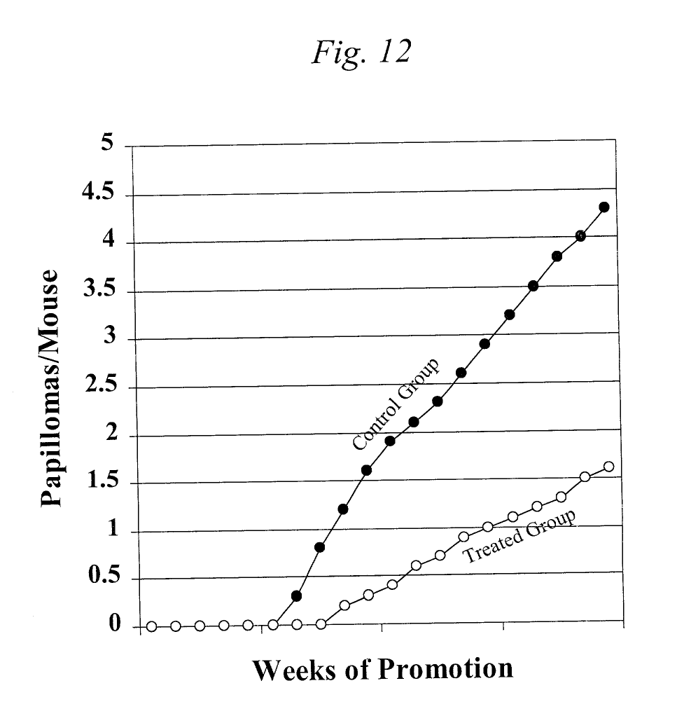

In a third experiment tumors were initiated in mice with DMBA (390 nmol) and promoted with eight minute exposures of 3430 J/m² UVB light given twice weekly starting 1 week after initiation. Mice to be treated were given 50 μg of Agaricus aqueous extract by topical application 1 hour prior to exposure to the UVB light source and compared to control mice initiated and promoted in the same way but given no extract (FIGS. 9 and 10). Table 6, below and FIGS. 11 and 12 show the inhibitory effects of orally administered Agaricus aqueous extract on tumor proliferation.

TABLE 6
Test Results for Topical Treatment of TPA Promoted Mice

| Positive control | | | 50 μg Agaricus extract | |
|---|---|---|---|---|
| DMBA (390 nmol) + UVB (2x/week) | | | (topical) | |
| | | Papillomas/ | | Papillomas/ |
| Week | Papillomas (%) | Mouse | Papillomas (%) | Mouse |
| 1 | 0 | 0 | 0 | 0 |
| 2 | 0 | 0 | 0 | 0 |
| 3 | 0 | 0 | 0 | 0 |
| 4 | 0 | 0 | 0 | 0 |
| 5 | 0 | 0 | 0 | 0 |
| 6 | 0 | 0 | 0 | 0 |
| 7 | 20 | 0.3 | 0 | 0 |
| 8 | 60 | 0.8 | 0 | 0 |
| 9 | 80 | 1.2 | 20 | 0.2 |
| 10 | 86.6 | 1.6 | 20 | 0.3 |
| 11 | 100 | 1.9 | 20 | 0.4 |
| 12 | 100 | 2.1 | 40 | 0.6 |
| 13 | 100 | 2.3 | 40 | 0.7 |
| 14 | 100 | 2.6 | 46.6 | 0.9 |
| 15 | 100 | 2.9 | 60 | 1.0 |
| 16 | 100 | 3.2 | 66.6 | 1.1 |
| 17 | 100 | 3.5 | 73.3 | 1.2 |
| 18 | 100 | 3.8 | 80 | 1.3 |
| 19 | 100 | 4.0 | 86.6 | 1.5 |
| 20 | 100 | 4.3 | 86.6 | 1.6 |

As the results above show, at 20 weeks of promotion, controls significantly differed from the treated group in papillomas per mouse (p>0.05) and in the number of mice showing the presence of papillomas

The preceding description and examples are intended to be illustrative. For example, although the above dosages are given for treatment and prevention of tumors in animals, it will be understood that these dosages can be easily converted to human dosages through well know body weight differentiation calculations. In this calculation the weight of the test animal and the patient are ratioed and the dosage corrected accordingly. In this case the average weight of a mouse used in the above experiments is 30 g, while the average weight of a human is 60 kg, a weight difference of 2000. Accordingly, the dosages reported above can be easily converted to humans by taking the effective amount given to the mice and converting it to the dosage required to provide a similar effect for the larger mass of the human body.

For example, in the oral feeding dosage a mouse consumes 7 mL of Agaricus aqueous extract having a dilution ratio of 2.5 mg/100 mg. During the course of a twenty week treatment, an average mouse took 24.5 mg of Agaricus extract. Converting this dosage to humans yields a dosage of about 49 g of Agaricus or approximately 122 mg twice a week.

In the topical application atypical mouse received 50 μg of Agaricus extract for 20 weeks. Converting this dosage to humans yields a total dosage of 4 g of Agaricus extract, or approximately two applications of 100 mg of Agaricus each week.

Those skilled in the art to which the invention pertains will appreciate that alterations and changes in the described protocols may be practiced without departing from the meaning, spirit, and scope of this invention. Therefore, the foregoing description should be read to have its fullest and fair scope.

What is claimed is:

1. A method of preventing and treating a skin disorder comprising the steps of:
providing a composition comprising a quantity of Agaricus blazei Murill; and
administering an effective amount of said composition to a subject.

2. A method as described in claim 1, wherein the composition is administered orally.

3. A method as described in claim 1, wherein the composition is formulated in a liquid solution.

4. A method as described in claim 1, wherein the composition is in a form selected from the group consisting of a powder, crystalline and granule.

5. A method as described in claim 1, wherein the composition is administered topically.

6. A method as described in claim 1, wherein the composition is formulated as one of the group consisting of: a cream, ointment, gel, cosmetic lotion and a shampoo.

7. A method as described in claim 1, wherein the Agaricus blazei Murill is present in a concentration of at least about 10 mg per unit composition.

8. A method as described in claim 1, wherein the Agaricus blazei Murill is present in a concentration of between about 10 mg and 600 mg per unit composition.

9. A method as described in claim 1, wherein the Agaricus blazei Murill is present in a concentration of about 100 mg per unit composition.

10. A method as described in claim 1, wherein the Agaricus blazei Murill is an extract obtained by water extraction.

11. A method as described in claim 1, wherein the Agaricus blazei Murill is an extract obtained according to the following steps:
providing whole or particulate Agaricus blazei Murill;
extracting the whole or particulate Agaricus blazei Murill in hot water to obtain a residue;
extracting the residue in an aqueous solution of ammonium oxalate to obtain an extract;
decomposing the extract in hydrochloric acid;
gel permeating the extract; and
purifying the extract with affinity chromatography.

12. A method as described in claim 10 or 11, wherein the extract is at least one substance selected from the group consisting of: a polysaccharide-glucan, a steroid, a dietary fiber; linoleic acid; ergosterol; nicotinic acid amide; benzoic acid and beta glucans.

13. A method as described in claim 1, wherein the composition is formulated as a pill or capsule.

14. A method as described in claim 1, wherein the composition further comprises other active or inactive ingredients.

15. A method as described in claim 1, wherein the skin disorder is one selected from the group consisting of: neoplasms, melanoma, multicentric primary skin malignancy, and viral skin disease.

16. A method as described in claim 1, wherein the skin disorder is giant congenital melanocytic nevi.

17. A method as described in claim 1, wherein the skin disorder is either a basal cell or squamous cell cancer.

18. A method as described in claim 1, wherein the skin disorder is either mycosis fungoides or Kaposi's sarcoma.

19. A method as described in claim 1, wherein the skin disorder is either viral warts or molluscum contagiosum.

20. A method as described in claim 1, wherein the Agaricus blazei Murill is an extract and is extracted at a ratio of about one part Agaricus blazei to about 2 to 5 parts aqueous solution.

21. A method as described in claim 1, wherein the composition contains at least about 0.01% Agaricus blazei Murill by volume.

22. A method as described in claim 1, wherein the composition contains at least about 0.05% Agaricus blazei Murill by volume.

23. A method as described in claim 1, wherein the composition contains at least about 0.05% to about 20% Agaricus blazei Murill by volume.

24. A method as described in claim 1, wherein the subject is a domesticated or livestock animal.

25. A method as described in claim 1, wherein the subject is a human being.

26. A method as described in claim 1, wherein the composition is administered at least once daily for between about 10 to about 30 days.

27. A method as described in claim 1, wherein an interval of between about 1 and about 10 days is provided between administrations of the compound.

28. A method as described in claim 1, wherein the composition is formulated into either an animal food or a dietary supplement for animals

29. A method as described in claim 1, wherein the Agaricus blazei Murill is in a form selected from the group consisting of: whole,. particulate and extract thereof.

30. A method as described in claim 1, wherein the composition comprises Agaricus blazei Murill from all four stage of the mushroom lifecycle.

31. A method of preventing and treating an autoimmune disorder comprising the steps of:
providing a composition comprising Agaricus blazei Murill; and
administering an effective amount of said composition to a subject.

32. A method as described in claim 31, wherein the composition is administered orally.

33. A method as described in claim 31, wherein the composition is formulated in a liquid solution.

34. A method as described in claim 31, wherein the composition is in a form selected from the group consisting of a powder, crystalline and granule.

35. A method as described in claim 31, wherein the composition is administered topically.

36. A method as described in claim 31, wherein the composition is formulated as one of the group consisting of: a cream, ointment, gel, cosmetic lotion and a shampoo.

37. A method as described in claim 31, wherein the Agaricus blazei Murill is present in a concentration of at least about 10 mg per unit composition.

38. A method as described in claim 31, wherein the Agaricus blazei Murill is present in a concentration of between about 10 mg and 600 mg per unit composition.

39. A method as described in claim 31, wherein the Agaricus blazei Murill is present in a concentration of about 100 mg per unit composition.

40. A method as described in claim 31, wherein the Agaricus blazei Murill is an extract obtained by water extraction.

41. A method as described in claim 31, wherein the Agaricus blazei Murill is an extract obtained according to the following steps:
providing whole or particulate Agaricus blazei Murill;
extracting the whole or particulate Agaricus blazei Murill in hot water to obtain a residue;
extracting the residue in an aqueous solution of ammonium oxalate to obtain an extract;
decomposing the extract in hydrochloric acid;
gel permeating the extract; and
purifying the extract with affinity chromatography.

42. A method as described in claims 40 or 41, wherein the extract is at least one substance selected from the group consisting of: a polysaccharide-glucan, a steroid, a dietary fiber; linoleic acid; ergosterol; nicotinic acid amide; benzoic acid and beta glucans.

43. A method as described in claim 31, wherein the composition is formulated as a pill or capsule.

44. A method as described in claim 31, wherein the composition further comprises other active or inactive ingredients.

45. A method as described in claim 31, wherein the autoimmune disorder is one selected from the group consisting of: intravascular disorders, immune disorders and neuromuscular disorders.

46. A method as described in claim 31, wherein the autoimmune disorder is selected from the group consisting of: hemolytic anemia, thyroid goiter, Hashimoto's disease, pernicious anemia, type I and II diabetes mellitus and chronic hepatitis.

47. A method as described in claim 31, wherein the autoimmune disorder is selected from the group consisting of: gloerulonephritis, systemic lupus erythematosus and Sjongren's disease.

48. A method as described in claim 31, wherein the autoimmune disorder is selected from the group consisting of: myasthenia gravis and Graves' disease.

49. A method as described in claim 31, wherein the autoimmune disorder is a disease selected from the group consisting of: rheumatoid arthritis, multiple schlerosis and ulcerative colitis.

50. A method as described in claim 31, wherein the Agaricus blazei Murill is an extract and is extracted at a ratio of about one part Agaricus blazei to about 2 to 5 parts aqueous solution.

51. A method as described in claim 31, wherein the Agaricus blazei Murill is an extract and is extracted at a ratio of about one part Agaricus blazei to between about 25 to 100 parts aqueous solution.

52. A method as described in claim 31, wherein the composition contains at least about 0.01% Agaricus blazei Murill by volume.

53. A method as described in claim 31, wherein the composition contains at least about 0.05% Agaricus blazei Murill by volume.

54. A method as described in claim 31, wherein the composition contains at least about 0.05% to about 20% Agaricus blazei Murill by volume.

55. A method as described in claim 31, wherein the subject is an animal.

56. A method as described in claim 31, wherein the subject is a human being.

57. A method as described in claim 31, wherein the composition is administered at least once daily for between about 10 to about 30 days.

58. A method as described in claim 31, wherein an interval of between about 1 and about 10 days is provided between administrations of the compound.

59. A method as described in claim 31, wherein the composition is formulated into either an animal food or a dietary supplement for animals

60. A method as described in claim 31, wherein the Agaricus blazei Murill is in a form selected from the group consisting of: whole, particulate and extract thereof.

61. A method as described in claim 31, wherein the composition comprises Agaricus blazei Murill from all four stage of the mushroom lifecycle.

62. A method of preventing and treating a skin disorder comprising the steps of:
providing a composition comprising an Agaricus blazei Murill; and
administering at least about 10 mg of said composition per day to a subject over a period of between about 10 and 30 days.

63. A method as described in claim 62, wherein an interval of between about 1 and about 10 days is provided between administrations of the compound.

64. A method as described in claim 62, wherein the composition is administered to the subject either orally or topically.

65. A method of preventing and treating an autoimmune disorder comprising the steps of:
providing a composition comprising an Agaricus blazei Murill; and
administering at least about 10 mg of said composition per day to a subject over a period of between about 10 and 30 days.

66. A method as described in claim 65, wherein an interval of between about 1 and about 10 days is provided between administrations of the compound.

67. A method as described in claim 65, wherein the composition is administered to the subject either orally or topically.
